(19) United States
(12) Patent Application Publication
(10) Pub. No.: US 2012/0022480 A1
(43) Pub. Date: Jan. 26, 2012

(54) DISPOSABLE ABSORBENT DEVICE FOR POST-URINARY DRIP AND DISPENSER FOR THE DEVICE

(76) Inventor: Gary J. Trabucco, New Windsor, NY (US)

(21) Appl. No.: 12/841,264

(22) Filed: Jul. 22, 2010

(51) Int. Cl. A61F 13/471; F16M 13/02; B65D 83/00; A61L 15/20; A61F 13/60

(52) U.S. Cl. 604/377; 604/385.03; 604/385.05; 221/36; 248/205.1

(57) ABSTRACT

A disposable absorbent device for post-urinary drip has a sanitary shield or pad formed as a curved shell configured to substantially conform to the exterior surface of the penis glans, said shield or pad defining interior concave and exterior convex surfaces. A fluid permeable layer of sheet material lines at least a central portion of the interior surface of the shield or pad. Adhesive covered tabs about the periphery of the shell detachably secures the shield on the glands to provide contact with the fluid permeable layer. A fluid impermeable layer is provided on at least a portion of the exterior surface so that a disposable absorbent device may be secured to the glans for covering the urethral opening in the glans for capturing and retaining any fluid drip post urination or post sexual activity with the shield without manipulation or physical transformation of the device prior to application, while providing a virtual barrier to reduce exposure of the glans from fluid absorbed by said shield. A dispenser for a disposable hygienic device formed as a cylindrical container having a cylindrical wall, a closed bottom wall and top opening. The container allows devices to freely move from the bottom wall to the top opening. A support member within the container supports a plurality of stacked devices for advancing a device at the top of a stack to the top opening. The support member includes tabs for manual application of an upward force to the support member for a user to elevate the support member and position an upper most device to the top opening for manual removal from the stack.

### BACKGROUND OF THE INVENTION

1. Field of the Invention

The present invention generally relates to the field of hygienic or sanitary devices and, more particularly, the invention relates to a disposable absorbent sanitary device specifically configured to be comfortably applied to the penis glans to capture and absorb post-urinary drip, and a dispenser for dispensing such devices.

2. Description of the Prior Art

The inability to control one's bodily functions is a public health problem, affecting an enormous number of people. When a male is unable to control the flow of urine, this condition is known as urinary incontinence. The sanitary paper products industry sells products such as adult diapers to treat people with this condition. However, there are many degrees of incontinence, and many males suffer from only minor leakage, in which they drip small amounts of urine following urination. This condition is commonly referred to as “post-urinary drip”. For these people, adult diapers and similar items, which are bulky and uncomfortable, are impractical. Yet, post-urinary drip continues to be a problem because it creates an unsanitary condition that can spread contagious diseases and can also contact and soil portions of apparel such as shorts, briefs or other underpants that absorb and are wetted by the urine. Because post-urinary drip can also wet through underpants it can cause spotting of outergarments and embarrassment to afflicted males.

Devices that absorb post-urinary drip but which aim to be less bulky and uncomfortable, have been developed in the past. Devices such as those disclosed in U.S. Patent Application Publication Nos. 2007/0262087; 2004/0059308; U.S. Pat. Nos. 6,613,954; 6,479,726; 4,863,448; 4,795,450; 4,668,229; 4,601,716; 4,576,599 and 4,500,314 address these concerns. These patents, however, present difficulties in that they provide improvement in this regard but are still generally bulky and uncomfortable. In addition the devices described are difficult and inconvenient to use and to dispose of.

Another problem inherent with most if not all of the devices described in the aforementioned patent publications is that they must be physically altered from a first state, typically in a flat two-dimensional condition, in which they are stored, and a second state in which they are expanded into a three-dimensional form suitable for use. However, such physical alteration requires added work and inconvenience and requires folding or bending portions of the device that typically resist such alterations or deformations. This results in restoring forces that tend to urge the altered shape to revert to the original two-dimensional shape or state. Such restoring forces must be compensated for by adhesive portions that attach the altered form of the device to the glans. Such restoring forces, therefore, require additional adhesion to overcome the restoring forces and retain the device in place. However, if adhesive forces are too great, removal can be a problem. Yet if the adhesive forces are not adequate, the device can inadvertently and undesirably separate from the skin and thereby defeat the objectives for using the device.

Another problem with known post-urinary drip appliances or devices is that they have not been provided with suitable dispensers that can be placed proximate to urinals in men's public restrooms thereby depriving men of convenient access to such devices. This has prevented the widespread use of post-urinary drip sanitary devices.

An improved device and dispenser are needed specifically designed to be hygienic, simple and straightforward to apply and use, and also be accessible and comfortable. Such device must also be easy to remove and dispose of.

### SUMMARY OF THE INVENTION

It is therefore an object of the present invention to provide a new and improved device that absorbs post-urinary drip, and that pulls wetness away from the body, enabling the wearer to remain comfortable and dry, and protects the wearer's clothes.

It is a further object of the present invention to provide a new and improved device that can absorb post-urinary drip to promote public health by reducing potential spread of contagious diseases.

It is a further object of the present invention to provide a new and improved device that can absorb post-urinary drip that can be readily accessed from a dispenser.

It is a further object of the present invention to provide a new and improved device that can absorb post-urinary drip and is convenient to apply, use and dispose.

It is a further object of the present invention to provide a new and improved device that can absorb post-urinary drip and bodily fluids after sexual activity and is lightweight and comfortable to apply and wear.

It is a yet further object of the present invention to provide a new and improved disposable device that can absorb post-urinary drip and that can be manufactured inexpensively and can be affordably priced.

It is still a further object of the present invention to provide a post-urinary drip device that can be applied as dispensed without requiring any manipulations or transformations of the device prior to application.

It is an additional object of the present invention to provide a dispenser for the devices in accordance with the invention.

It is yet an additional object of the present invention to provide a device and dispenser that can be dispensed in men's public restrooms.

It is still a further object of the present invention to provide a post-urinary drip device that is simple to use and can be retrieved and applied with one hand.

It is a further additional object of the present invention to provide a post-urinary drip device that is effective to protect the wearer's clothes from spotting due to post-urinary drip.

In order to achieve the above objects, as we as others that become evident hereinafter, a disposable absorbent device for post-urinary drip comprises a sanitary shield or pad formed as a generally spherical shell, configured to substantially conform to the exterior surface of the penis glans. The shield or pad defines lines generally spherical interior and exterior surfaces. A fluid permeable layer of sheet material lines at least a central portion of the interior surface of said shield or pad. Means is provided for detachably securing such shield or pad to the skin of the penis glans of a user to provide contact with said permeable layer. A fluid impermeable layer is provided on at least a portion of said exterior surface. In this manner, a disposable absorbent device may be secured to the glans for covering the urethral opening in the glans for capturing and retaining any fluid post urination or post-sexual activity within such shield or pad without manipulating or physically transforming the device prior to application, while providing a virtual barrier to reduce exposure of the glans from fluid absorbed by such shield or pad.

A dispenser in accordance with the invention for dispensing disposable hygienic devices is formed as a generally spherical shell to receive a stack of devices that define a predetermined periphery. The dispenser comprises a generally cylindrical container having a cylindrical wall defining a generally vertical axis when mounted on a wall or other support surface. The container has a closed bottom wall and a top opening. Said cylindrical container has a generally uniform cross-section along said vertical axis in parallel horizontal planes. Said predetermined cross-section generally corresponds to the shape and size of said predetermined periphery of the devices to allow individual devices to freely move along said axis from said bottom wall to said top opening. A support member within said container is movable along said axis for supporting a plurality of stacked devices and for advancing a device at a top of the stack to said top opening. Said support member includes gripping means extending exteriorly of said cylindrical wall for manual application of an upward force to said support member for a user to elevate said support member and position an uppermost device proximate to the top opening for manual removal of a single device from the stack by lifting it off the stack along the direction of said axis.

### BRIEF DESCRIPTION OF THE DRAWINGS

Those skilled in the art will appreciate the improvements and advantages that derive from the present invention upon reading the following detailed description, claims, and drawings, in which:

FIG. 1 is a side elevational view of an absorbent device for post-urinary drip in accordance with the invention;

FIG. 2 is an exploded side elevational view of the device shown in FIG. 1;

FIG. 3 is a perspective view of the device shown in FIGS. 1 and 2 with the device in its concave surface upward orientation;

FIG. 4 is a top plan view of the device shown in FIGS. 1-3;

FIG. 5 is a perspective view of a dispenser for disposing disposable absorbent devices of the type shown in FIGS. 1-4, a stack of such devices being shown with the upper most device in the stack in a position to be lifted and removed from the dispenser; and

FIG. 6 is a view similar to FIG. 5, with a portion of the cylindrical wall removed to show details of the a movable support member that supports a stack of devices and that can be manually gripped outside of the dispenser to raise a stack of devices in order to position the uppermost device in the stack at the open end of the dispenser for ready access and removal.

### DESCRIPTION OF THE PREFERRED EMBODIMENTS

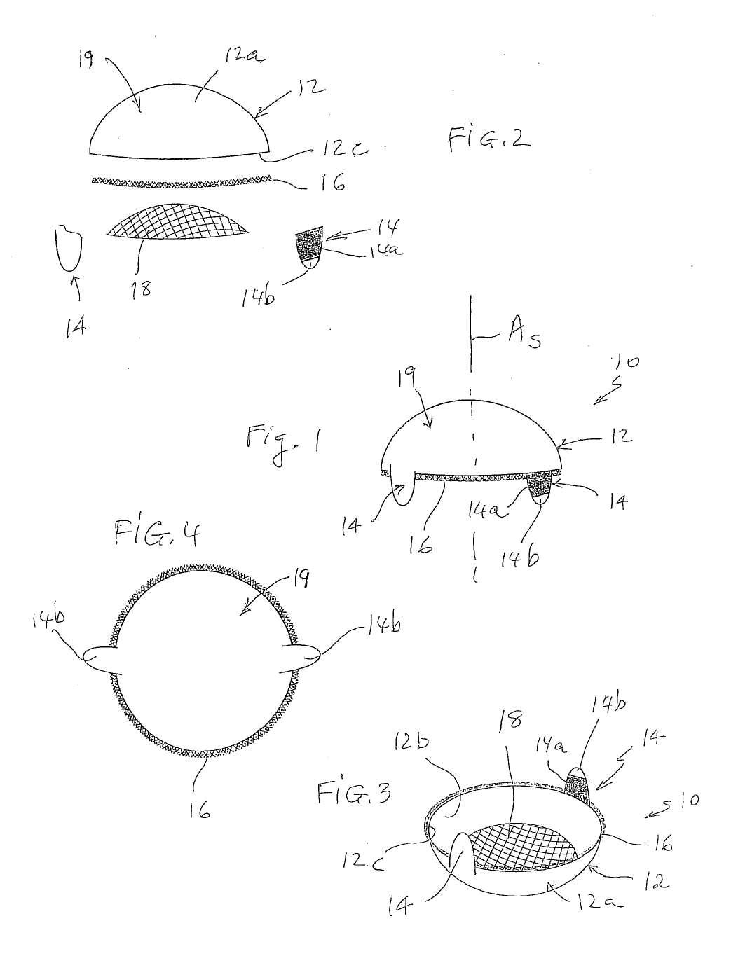

Referring now specifically to the Figures, in which identical or similar parts will be designated by the same reference numerals throughout, and first referring to FIGS. 1-4, a disposable absorbent device in accordance with the invention is generally designated by the reference numeral 10.

The device 10 includes a sanitary shield or pad 12 formed as a generally spherical shell and configured to substantially conform to the exterior surface of the penis glans. It will be appreciated, however, that the specific configuration may deviate from a perfectly spherical shell, the objective being to substantially conform with the exterior surface of the glans. In this way, the device 10 can be applied to the glans without physical transformation or modification of the shape or configuration of the device prior to application.

The pad 12 is formed of a highly absorbent material. The absorbent material may be composed of natural fiber such as cotton or artificial polymer and may contain absorbent gel material. Other embodiments of the device may contain varied thicknesses of absorbent material so that the product may be sold in thinner, less-absorbent and thicker, more absorbent embodiments to allow the user to select an appropriate product to address the severity of the post-urinary drip problem.

The shell 12 forms an exterior convex surface 12a and an interior concave surface 12b as best shown in FIGS. 2 and 3 and defines a predetermined periphery 12c which, in the embodiment shown, is circular. A pair of flexible tabs 14 are provided on diametrically opposite sides of the shell. The shell defines an axis Aₛ, the tabs 14 extending downwardly beyond the shell periphery in a direction generally parallel to the axis Aₛ. Each tab 14 has an adhesive portion 14a and a non-adhesive portion 14b that allows a user to grip the tabs 14 for removal from a dispenser, to be described, and from the glans when no longer needed. The adhesive portions 14a face inwardly as shown, so that the adhesive surfaces can be attached to the skin of the glans post urination or post-sexual activity. If desired the adhesive portions 14a may be initially covered with removable backing that can be peeled off just prior to use similar to backing used with conventional adhesive bandages, such as adhesive bandages sold under the trademark BAND AID®.

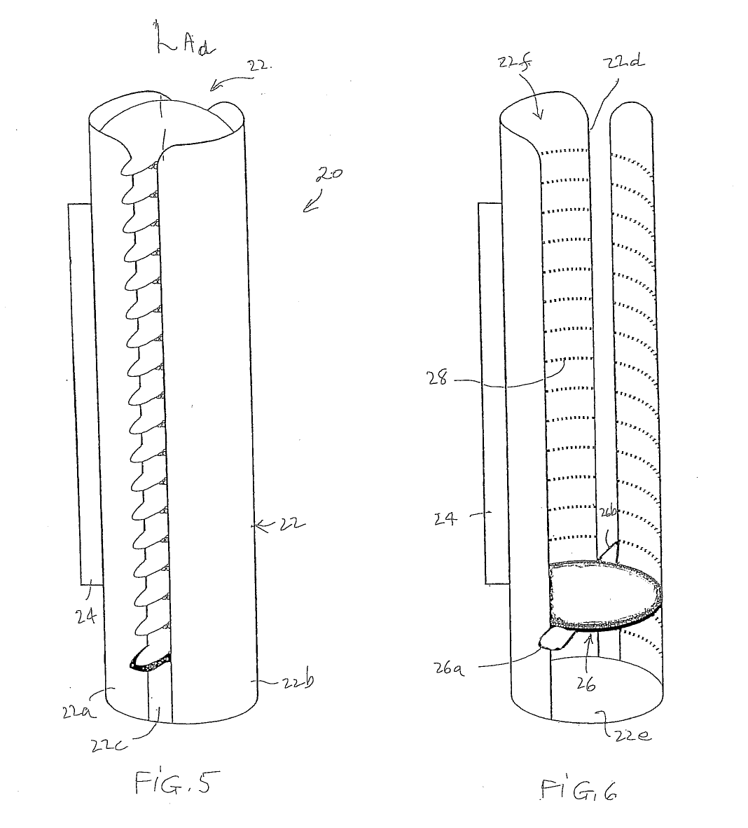

In FIG. 3 the tabs 14 are shown in their inner operative positions when secured to the glans, while in FIG. 4 they are shown extending radially outwardly—their initial orientations while in a dispenser, as to be described.

Advantageously, but optionally, a gentle elastic 16 is provided, formed about the perimeter 12c to help the device to stay in place, once the tabs have been secured.

A fluid permeable layer sheet 18 lines at a least a central portion of the interior surface 12b of the shield or pad, generally symmetrically arranged about the axis Aₛ. In the presently preferred embodiment, the fluid permeable layer 18 is an open weave mesh that separates the surface of the glans from the absorbent shell or pad 12. After the fluid has penetrated the barrier 18 and has been absorbed within the shield or pad, the layer 18 serves as a spacer that effectively forms as a barrier to minimize or eliminate contact between the skin of the glans and the shield or pad that has absorbed and stores the excess fluid.

The adhesive portions 14a may incorporate the same or similar adhesives as used on adhesive bandages and should provide sufficient adhesion to prevent inadvertent removal or separation of the device from the glans. Since the device is shaped to conform to the shape of the glans, the device does not need to be deformed in any way and, therefore, the natural configuration or shape of the device 10 requires very little adhesive forces to retain the device.

A fluid impermeable layer 19 is provided on at least a portion of the exterior surface 12a of the shell 12 to serve as a barrier to the fluid that has been absorbed within the shell to thereby prevent any fluid stored within the absorbent shell material from coming into a contact with other surfaces on the convex surface 12a side of the device.

It will be appreciated that a disposable absorbent device 10 in accordance with the invention may be secured to the glans for covering the urethral opening in the glans and for capturing and retaining any fluid post drip urination or post sexual activity within a shield or pad without any physical transformation or deformation of the device prior to application the device. Therefore, it is extremely simple in construction and simple to use. Removal of the device merely requires the application of sufficient force to break the adhesion between the relatively small adhesive area 14a and the equally small area of the skin to which the portions 14a are adhered.

Referring now specifically to FIGS. 5 and 6, a dispenser in accordance with the invention specifically configured to dispense the disposable absorbent devices shown in FIGS. 1-4 is generally designated by the reference numeral 20. The dispenser 20 is specifically adapted to dispose the hygienic devices 10 and is formed as generally spherical shells, from a stack of shells each of which defines a predetermined periphery 12c. The dispenser 20 is formed as a generally cylindrical container 22 having a cylindrical wall that defines a generally vertical axis A_{d} when mounted on a wall or other support surface.

The container or dispenser 22 is formed of two semi-cylindrical juxtaposed shells 22a, 22b that are substantially parallel to each other as shown to define an internal cylindrical surface and diametrically opposite slots 22c, 22d. The container has a bottom wall 22e and a top opening 22f as shown.

The container 22 has a generally uniform cross-section along the vertical axis A_{d} along its longitudinal length. In the embodiment illustrated the cross-section is circular although it will be appreciated that slight deviations from the circular shape can be used, with different degree of advantage. Importantly, the cross-sectional area of the container 22 needs to conform to periphery 12c of the device and be slightly greater than that periphery to provide a clearance to thereby permit the devices to be freely movable between the bottom wall 22 and the top opening 22f.

Any suitable bracket 24 may be secured to one of these semi-cylindrical shells 22a, 22b for attachment to a wall or other vertical surface.

According to one feature of the invention, a support member 26 is provided in the form of a disk or circular plate provided with diametrically outwardly extending tabs 26a, 26b each of which is dimensioned to extend through an associated slot 22c, 22d with clearance so that the tabs can be manually gripped by a user to lower and raise the support member 26 within the container while remaining in a generally horizontal orientation as shown in FIG. 6.

The support member 26 is adapted to support a plurality of stacked devices 10 one above the other, as suggested in FIG. 5. In this manner, raising or lifting the support member 26, with a stack of devices 10 supported thereon, elevates the stack and the support member to position an uppermost device 10 from the stack to be brought into proximity of the open end 22f for manual removal from the stack by a user. Since the devices are curved shells, a stack of such devices results in the devices being nestled one within the other and, unlike conventional dispensers, the devices cannot be pulled laterally or radially outwardly as with planar stacked units. Instead, an uppermost shell needs to be lifted in order to clear the stack and be separated from the stack and the next lower shell on which it is nestled.

Advantageously, the outer periphery of the support member 26 is provided with frictional material to provide a degree of friction within the internal surface of the dispenser or the walls of the shell members 22a, 22b. Alternatively, the internal surface of the container may be provided with a plurality of generally parallel vertically spaced detents 28 that allow the support member 26 to be snapped into place each time it is raised. It cooperates with a next successively higher detent 28 for maintaining the support member and the remaining devices stacked thereon at a suitable position to arrange the upper most shell in the stack in proximity to the open end 22f as shown in FIG. 5.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A disposable absorbent device for post-urinary drip comprising a sanitary shield or pad formed as a curved shell configured to substantially conform to the exterior surface of the penis glans, said shield or pad defining interior concave and exterior convex surfaces;
a fluid permeable layer of sheet material lining at least a central portion of said interior surface of said shield or pad;
means for detachably securing said shield or pad on the glands to provide contact with said fluid permeable layer; and
a fluid impermeable layer provided on at least a portion of said exterior surface whereby a disposable absorbent device may be secured to the glans for covering the urethral opening in the glans for capturing and retaining any fluid drip post urination or post sexual activity with said shield or pad without manipulation or physical transformation of the device prior to application while providing a virtual barrier to reduce exposure of the glans from fluid absorbed by said shield or pad.

2. A disposable absorbent device as defined in claim 1, wherein said sanitary shield is in the from of a cap or segment of a generally spherical shell having an axial or radial dimension that is less than the radius of the spherical shell.

3. A disposable absorbent device as defined in claim 1, wherein said sanitary shield is formed of natural fiber.

4. A disposable absorbent device as defined in claim 1, wherein said sanitary shield is formed of cotton.

5. A disposable absorbent device as defined in claim 1, wherein said sanitary shield is formed of an artificial polymer.

6. A disposable absorbent device as defined in claim 1, wherein said sanitary shield incorporates absorbent gel.

7. A disposable absorbent device as defined in claim 1, wherein said means comprises a pair of diametrically opposite tabs along a peripheral edge of said shield or pad that are accessible and include adhesive means for selectively attaching and detaching said shield or pad.

8. A disposable absorbent device as defined in claim 7, wherein said tabs are initially provided with removable backing material for exposing the adhesive only after said backing material is removed.

9. A disposable absorbent device as defined in claim 1, wherein said fluid permeable layer comprises an open weave mesh material.

10. A disposable absorbent device as defined in claim 1, wherein said fluid impermeable layer comprises a thin sheet of plastic material.

11. A disposable absorbent device as defined in claim 7, wherein at least two tabs are provided along said peripheral edge.

12. A dispenser for a disposable hygienic device formed as a generally spherical cap or segment of a spherical shell defining a predetermined periphery, comprising a generally cylindrical container having a cylindrical wall defining a generally vertical axis when mounted on a wall or other support surface, said container having a closed bottom wall and top opening, said cylindrical container having a generally uniform cross-section along said vertical axis in parallel horizontal planes, said predetermined cross-section generally conforming to the shape of said predetermined periphery of the device to allow individual hygienic devices to freely move along said axis from said bottom wall to said top opening; and
a support member within said container movable along said axis for supporting a plurality of stacked devices and for advancing a device at the top of a stack to said top opening, said support member including gripping means for manual application of an upward force to said support member for a user to elevate said support member and position an uppermost device to said top opening for manual removal from the stack through said top opening.

13. A disposable absorbent device as defined in claim 12, further comprising attachment means for securing said dispenser to a wall or other vertical support member.

14. A disposable absorbent device as defined in claim 13, wherein said attachment means comprises a bracket secured to said cylindrical wall suitable for attachment to a wall or other surface by means of fasteners.

15. A disposable absorbent device as defined in claim 12, wherein said support member comprises a generally flat disc having a periphery that generally conforms to said predetermined cross-section.

16. A disposable absorbent device as defined in claim 12, wherein said gripping means comprises a pair of tabs extending radially outwardly about said support member to extend through vertical slots within said cylindrical container.

17. A disposable absorbent device as defined in claim 12, further comprising securing means for securing said support member along selected axial vertical positions along said cylindrical container.

18. A disposable absorbent device as defined in claim 17, wherein said securing means comprises inter-acting frictional surfaces about said support member and at the interior surface of said cylindrical container.

19. A disposable absorbent device as defined in claim 17, wherein said securing means comprises detents about said support member and the interior surface of said cylindrical container.

20. A stack of disposable devices nestled to form a column suitable for insertion into a vertical dispenser comprising a generally cylindrical container having a cylindrical wall defining a generally vertical axis when mounted on a wall or other support surface, said container having a closed bottom wall and top opening to allow individual hygienic devices to freely move along said vertical axis from said bottom wall to said top opening; and
a support member within said container movable along said axis for supporting a plurality of stacked devices and for advancing a device at the top of a stack to said top opening, said support member including gripping means for manual application of an upward force to said support member for a user to elevate said support member and position an upper most device to said top opening for manual removal from the stack,
each device in the stack comprising a sanitary shield or pad formed as a generally curved shell configured to substantially conform to the exterior surface of the penis glans, said shield or pad defining interior concave and exterior convex surfaces;
a fluid permeable layer of sheet material lining at least a central portion of said interior surface of said shield or pad;
means for detachably securing said shield or pad on the glands to provide contact with said fluid permeable layer; and
a fluid impermeable layer provided on at least a portion of said exterior surface whereby a disposable absorbent device may be secured to the glans for covering the urethral opening in the glans for capturing and retaining any fluid drip post urination or post sexual activity with said shield or pad without manipulation or physical transformation of the device prior to application while providing a virtual barrier to reduce exposure of the glans from fluid absorbed by said shield or pad.
